(19) United States
(12) Patent Application Publication
(10) Pub. No.: US 2022/0323036 A1
(43) Pub. Date: Oct. 13, 2022

(54) IMAGE PROCESSING APPARATUS, METHOD, AND STORAGE MEDIUM

(71) Applicant: CANON KABUSHIKI KAISHA, Tokyo (JP)

(72) Inventor: Shinya Katsumata, Kanagawa (JP)

(21) Appl. No.: 17/809,462

(22) Filed: Jun. 28, 2022

Related U.S. Application Data
(63) Continuation of application No. PCT/JP2021/002422, filed on Jan. 25, 2021.

(30) Foreign Application Priority Data
Feb. 7, 2020 (JP) 2020-020073

(51) Int. Cl. A61B 6/00; A61B 6/03

(52) U.S. Cl. CPC A61B 6/5282; A61B 6/032; A61B 6/4291

(57) ABSTRACT

An image processing apparatus obtains target grid feature information indicating primary radiation transmittance and scattered ray transmittance of a target grid, and imaging grid feature information indicating primary radiation transmittance and scattered ray transmittance of an imaging grid that is used for imaging, and obtains a photographed image obtained through radiography that uses the imaging grid. The image processing apparatus estimates a scattered dose based on a relation between the photographed image, a primary radiation image, and a scattered ray image, the relation being expressed using the primary radiation transmittance and the scattered ray transmittance indicated by the imaging grid feature information, and adjusts a scattered dose of the photographed image based on the estimated scattered dose, the target grid feature information, and the imaging grid feature information.

### CROSS-REFERENCE TO RELATED APPLICATIONS

This application is a Continuation of International Patent Application No. PCT/JP2021/002422, filed Jan. 25, 2021, which claims the benefit of Japanese Patent Application No. 2020-020073, filed Feb. 7, 2020, both of which are hereby incorporated by reference herein in their entirety.

### BACKGROUND OF THE INVENTION

### Field of the Invention

The present invention relates to an image processing apparatus, a method, and a medium for processing an image obtained using radiation.

### Background Art

X-ray imaging apparatuses have been widely used in many fields for medical images, industrial non-destructive tests, and the like. In recent years, digital X-ray imaging apparatuses that use what is known as a “Flat Panel Detector” (hereinafter, abbreviated as an “FPD”) in which a large number of semiconductor elements for converting radiation into electrical signals are disposed in a two-dimensional matrix have been in widespread use.

When an image of an object is captured by an X-ray imaging apparatus, X-rays that are incident onto the FPD are divided mainly into primary X-rays that travel in a straight line from an X-ray source and reach the FPD, and secondary X-rays that are X-rays reaching the FPD after the direction thereof changes within an object due to the Compton effect (hereinafter, referred to as “scattered rays”). An image that is obtained from the primary X-rays is an image that is desired to be observed essentially, and the scattered rays, which are X-rays incident onto the FPD after the direction thereof changes, degrade the contrast of an image formed by the primary X-rays. In order to remove such scattered rays, in X-ray imaging, an instrument called a scattered ray grid (hereinafter, a “grid”) that shields scattered rays incident from a direction different from the X-ray focus direction using a grating of a lead foil that is open in the X-ray focus direction is commonly used. In addition, in recent years, scattered ray reduction processing for creating a high-contrast image like a photographed image obtained through imaging using a grid (hereinafter, a “grid-photographed image”), by capturing an image without using a grid, and estimating and reducing the scattered rays in a photographed image through image processing has also come into use.

In X-ray imaging that uses a grid (hereinafter, grid imaging), there has been an issue that an obtained image has biased values (hereinafter, shading) depending on the positional relation with an X-ray focus. In addition, there has been an issue that, in scattered ray reduction processing, the dose of radiation that reaches the FPD is larger than that of grid imaging due to absence of a grid, and, as a result, quantum noise also increases, thus deteriorating the image granularity.

In view of this, techniques that use both scattered ray reduction processing and imaging that uses a grid in which the ratio of the length in the X-ray transmission direction of a lead foil to the distance between strips in the grid of the lead foil is smaller than commonly used grids (hereinafter, a “low ratio grid”) have been developed (see Patent Documents 1 and 2). In X-ray imaging that uses a low ratio-grid, shading is less likely to occur than X-ray imaging that uses an ordinary grid, and, furthermore, the dose of radiation that reaches the FPD is smaller than imaging that does not use any grid, thus providing an advantage that quantum noise can also be reduced. On the other hand, there is the issue that, in X-ray imaging that uses a low ratio grid, scattered rays incident from a direction other than the direction of the X-ray focus are unlikely to be shielded, and thus the contrast of an obtained image decreases. A technique that uses both a low ratio grid and scattered ray reduction processing is a technique for compensating for a low contrast by performing scattered ray reduction processing while maintaining the advantage of the low ratio grid.

### CITATION LIST

### Patent Literature

### PTL1: Japanese Patent Laid-Open No. 2014-113479

### PTL2: Japanese Patent Laid-Open No. 2016-172098

In scattered ray reduction processing, scattered ray estimation processing for estimating a scattered dose using a used imaging grid (low ratio grid) is performed, and the components of the scattered rays are reduced in an image based on the processing result. In scattered ray estimation processing, the features of an imaging grid and a target grid, such as a point-spread function of the imaging grid, kernels of the target grid and the imaging grid are used. However, there are a large variety of imaging grids that can be used for X-ray imaging. In conventional techniques that include Patent Document 1 and Patent Document 2, features of a target grid and an imaging grid are required for executing scattered ray estimation processing, and it has not been easy to handle all of the imaging grids that can be possibly used by the user. Therefore, when an attempt is made to use both a low ratio grid and scattered ray reduction processing, there are constraints on a low ratio grid that can be used as an imaging grid.

### SUMMARY OF THE INVENTION

The present invention provides a technique that makes it easier to use a large variety of imaging grids along with scattered ray reduction processing.

According to one aspect of the present invention, there is provided an image processing apparatus comprising: a first obtaining unit configured to obtain target grid feature information indicating primary radiation transmittance and scattered ray transmittance of a target grid, and imaging grid feature information indicating primary radiation transmittance and scattered ray transmittance of an imaging grid that is used for imaging; a second obtaining unit configured to obtain a photographed image obtained through radiography that uses the imaging grid; an estimating unit configured to estimate a scattered dose based on a relation between the photographed image, a primary radiation image, and a scattered ray image, the relation being expressed using the primary radiation transmittance and the scattered ray transmittance indicated by the imaging grid feature information; and an adjusting unit configured to adjust a scattered dose of the photographed image based on the scattered dose estimated by the estimating unit, the target grid feature information, and the imaging grid feature information.

According to another aspect of the present invention, there is provided an image processing method comprising: obtaining target grid feature information indicating primary radiation transmittance and scattered ray transmittance of a target grid, and imaging grid feature information indicating primary radiation transmittance and scattered ray transmittance of an imaging grid that is used for imaging; obtaining a photographed image obtained through radiography that uses the imaging grid; estimating a scattered dose based on a relation between the photographed image, a primary radiation image, and a scattered ray image, the relation being expressed using the primary radiation transmittance and the scattered ray transmittance indicated by the imaging grid feature information; and adjusting a scattered dose of the photographed image based on the estimated scattered dose, the target grid feature information, and the imaging grid feature information.

According to another aspect of the present invention, there is provided an obtaining unit configured to obtain a scattered dose of a photographed image obtained through radiography that uses an imaging grid, based on (a) target grid feature information indicating primary radiation transmittance and scattered ray transmittance of a target grid, and (b) imaging grid feature information indicating primary radiation transmittance and scattered ray transmittance of the imaging grid used for imaging.

According to another aspect of the present invention, there is provided an image processing method comprising: obtaining a scattered dose of a photographed image obtained through radiography that uses an imaging grid, based on (a) target grid feature information indicating primary radiation transmittance and scattered ray transmittance of a target grid, and (b) imaging grid feature information indicating primary radiation transmittance and scattered ray transmittance of the imaging grid used for imaging.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a diagram showing a configuration example of an X-ray imaging apparatus according to an embodiment of the present invention.

FIG. 2 is a flowchart illustrating image processing according to an embodiment of the present invention.

FIG. 3 is a diagram showing an exemplary functional configuration of scattered ray reduction processing according to an embodiment of the present invention.

FIG. 4 is a diagram showing an example of a method for obtaining scattered ray features.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

Note that an example will be described below in which X-rays are used as radiation, but radiation in the present invention is not limited to X-rays. Examples of radiation in the present invention include α-rays, β-rays, γ-rays, and the like that are beams made of particles (including photons) discharged due to radiation decay, and, in addition, beams having energy of the same or larger extent such as X-rays, particle beams, cosmic rays are also included.

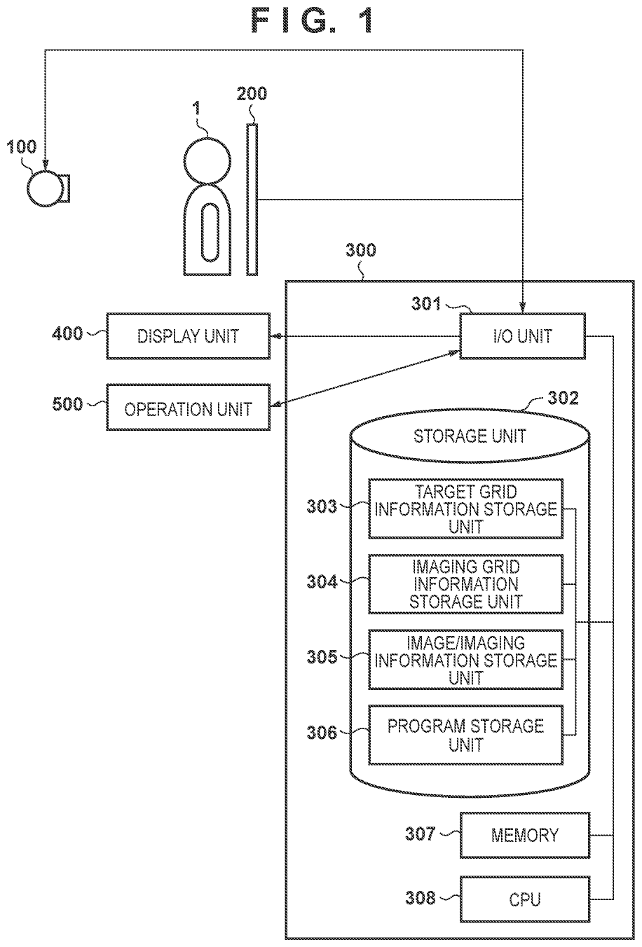

FIG. 1 is a diagram showing a configuration example of a radiography apparatus according to an embodiment of the present invention (hereinafter, an “X-ray imaging apparatus”). An X-ray tube 100 emits X-rays onto an object 1 and an FPD 200 on an extended line of the object 1. The FPD 200 irradiated with the X-rays converts the X-rays into an image, and transmits the image to an I/O unit 301 of an image processing apparatus 300. At this time, information regarding imaging at the time thereof (hereinafter, imaging information) such as a radiation dose and a tube voltage may be transmitted from the X-ray tube 100 to the image processing apparatus 300.

The I/O unit 301 of the image processing apparatus 300 functions as an interface with the X-ray tube 100, the FPD 200, a display unit 400, and an operation unit 500. The image processing apparatus 300 stores an image obtained from the FPD 200 via the I/O unit 301 and imaging information obtained from the X-ray tube 100, to an image/imaging information storage unit 305 of a storage unit 302. The stored image and imaging information can be used for scattered ray estimation processing, scattered ray adjustment processing, and the like, which will be described later. In addition, the storage unit 302 includes a target grid information storage unit 303 and an imaging grid information storage unit 304. The target grid information and the imaging grid information will be described later. A program storage unit 306 stores programs that have been loaded to a memory 307 and are to be executed by a CPU 308.

The memory 307 stores programs loaded from the storage unit 302 for the CPU 308 to execute the programs, and provides a work area for the CPU 308. The CPU 308 realizes various types of processing by executing programs stored in the program storage unit 306. Note that a computation device such as a GPU or an image processing chip may also be used instead of the CPU 308.

The display unit 400 performs various types of display under control of the image processing apparatus 300. The image processing apparatus 300 displays results of image processing, on the display unit 400, for example. In addition, the operation unit 500 is used for operations of the image processing apparatus 300, input of imaging information, input of target grid information and imaging grid information, and the like.

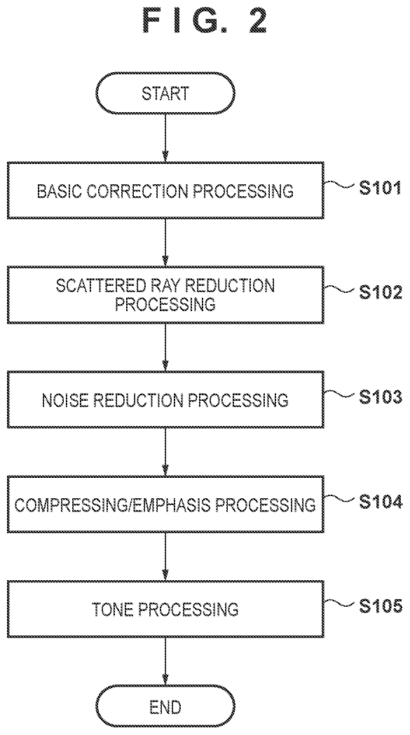

Next, image processing that is performed by the image processing apparatus 300 will be described with reference to the flowchart in FIG. 2. This image processing can be realized by the CPU 308 executing a predetermined program stored in the program storage unit 306 of the storage unit 302, for example. As a matter of course, as described above, the image processing may be realized by a dedicated computation device (hardware).

First, the CPU 308 corrects a device-specific feature of the FPD 200 regarding an image stored in the image/imaging information storage unit 305 of the storage unit 302 (step S101). Hereinafter, the processing of step S101 is referred to as “basic correction processing”. Specific processes of the basic correction processing includes gain correction for correcting variation in sensitivity between pixels, missing pixel correction for correcting a missing pixel based on the pixel values of surrounding pixels, offset correction for correcting pixel value components generated in an image due to a dark current flowing through an electronic circuit of the FPD 200, and the like.

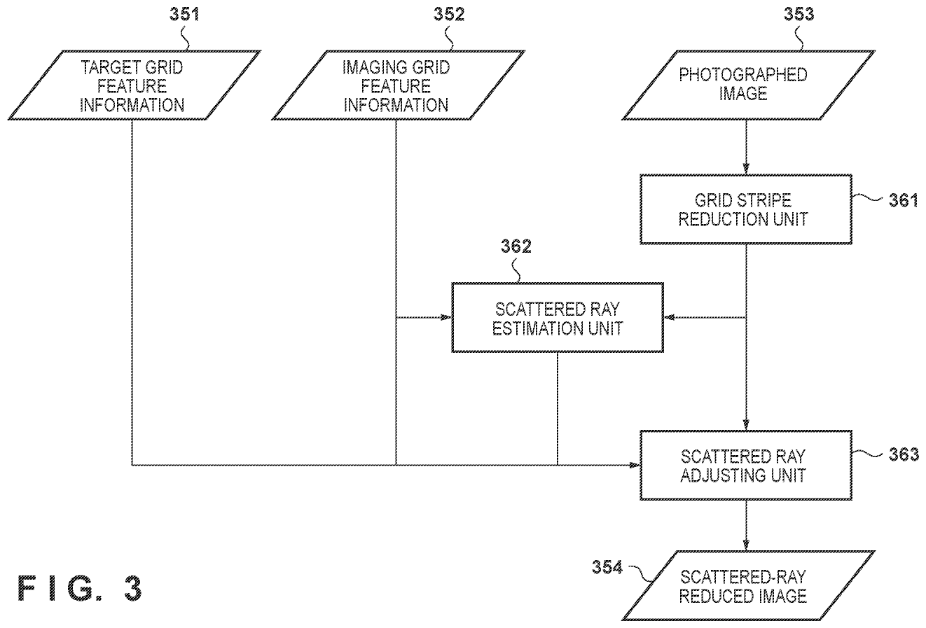

Next, the CPU 308 performs scattered ray reduction processing on the image subjected to the basic correction (step S102). The scattered ray reduction processing is processing for reducing the scattered dose in an image and improving the contrast to the contrast of a target grid image. Specific content of the scattered ray reduction processing will be described later with reference to FIG. 3. Next, the CPU 308 performs noise reduction processing for reducing the noise in an image, on the image subjected to the scattered ray reduction processing (step S103). A known noise reduction technique can be used for the noise reduction processing.

Next, the CPU 308 performs compressing/emphasis processing on the image subjected to the noise reduction processing (step S104). The compressing/emphasis processing aims to stabilize the luminance between images by performing compressing processing and to improve the visibility by performing emphasis processing. The CPU 308 performs compressing processing, for example, by separating high-frequency components and low-frequency components of the image from each other using a low-frequency filter, and decreasing the number of tones of the low-frequency components from the original number of tones, and performs emphasis processing for multiplying the high-frequency components by a coefficient to emphasize the components. The CPU 308 then performs tone processing on the image obtained in step S104 in order to improve the visibility of a final X-ray diagnosis image (step S105). The CPU 308 improves the contrast by increasing the number of tones of pixel values corresponding to the image of a diagnosis region, for example.

Next, the scattered ray reduction processing (step S102) will be described in detail with reference to the functional block diagram shown in FIG. 3. Note that some or all of the function units shown in FIG. 3 may be realized by the CPU 308 executing a predetermined program, or may also be realized by a dedicated computation device (hardware).

Target grid feature information 351 is held in the target grid information storage unit 303, and imaging grid feature information 352 is held in the imaging grid information storage unit 304. The target grid feature information 351 indicates the primary X-ray transmittance and the scattered ray transmittance of a target grid, and the imaging grid feature information 352 indicates the primary X-ray transmittance and the scattered ray transmittance of an imaging grid that is used for imaging. Primary X-ray transmittance and scattered ray transmittance are basic features of a grid defined in IEC60627Ed2. The target grid feature information 351 and the imaging grid feature information 352 are input from the operation unit 500 by the user, for example. In addition, a photographed image 353 subjected to the above-described basic correction processing (step S101), which is a photographed image obtained through X-ray imaging that uses an imaging grid, is held in the image/imaging information storage unit 305.

A grid stripe reduction unit 361 performs grid stripe reduction processing on the photographed image 353, and reduces the number of stripes of the grid in the photographed image 353. A scattered ray estimation unit 362 performs scattered ray estimation processing using the image subjected to grid stripe reduction processing and the imaging grid feature information 352, and obtains a scattered ray estimation image by estimating a scattered dose. A scattered ray adjusting unit 363 reduces the scattered dose so as to bring the contrast close to the image contrast of the target grid feature, based on the photographed image subjected to the grid stripe reduction processing, using the scattered ray estimation image, the target grid feature information 351, and the imaging grid feature information 352, and obtains a scattered-ray reduced image 354. The scattered-ray reduced image 354 is held in the image/imaging information storage unit 305, and is displayed on the display unit 400.

Next, the aforementioned function units that realize the scattered ray reduction processing will be described more in detail.

The target grid feature information 351 refers to the grid features as a target of an image contrast of the scattered-ray reduced image 354 that is output, and is used by the scattered ray adjusting unit 363. Here, the grid features refer to the primary radiation transmittance (hereinafter, primary X-ray transmittance) and the scattered ray transmittance of the grid. The target grid feature information 351 can be obtained by the user directly inputting grid feature information using the operation unit 500. Note that there is no limitation to this, and the target grid information may be indirectly obtained. A configuration may also be adopted in which, for example, the storage unit 302 or an external storage device stores the correspondence between each type of imaging grid and target grid feature information, and the image processing apparatus 300 selects target grid feature information in accordance with a type of imaging grid that is used for imaging. Alternatively, a configuration may also be adopted in which the correspondence between each imaging portion and target grid feature information is held in the storage unit 302 or an external storage device, and target grid feature information corresponding to an imaging portion input by the user from the operation unit 500 is selected by the image processing apparatus 300.

The imaging grid feature information 352 is grid feature information of the grid that is used for imaging, and is used by the scattered ray estimation unit 362 and the scattered ray adjusting unit 363. Similarly to the target grid feature information, the imaging grid feature information 352 can be directly input by the user via the operation unit 500. In addition, a configuration may also be adopted in which the image processing apparatus 300 can automatically identify an imaging grid to be used for imaging. The photographed image 353 is an image obtained after performing the basic correction processing (step S101) on an image captured using the imaging grid. The photographed image 353 may be stored in the storage unit 302, or may also be temporarily stored in the memory 307.

The grid stripe reduction unit 361 performs processing for reducing stripes that appear in an image based on the pixel size of the FPD 200 and slits in the lead foil for removing scattered rays in the grid (grid stripe reduction processing). This grid stripe reduction processing may be omitted if grid stripes are hardly visible or invisible depending on the relation between the grid density of the grid and the pixel size. A known technique can be used for the grid stripe reduction processing. Examples of such processing include a method for selecting a grid that causes stripes to appear at the high frequencies in the image based on the relation between the pixel pitch of the FPD 200 and the grid density of the grid, and removing the stripes by using a low-frequency filter in the photographed image.

The scattered ray estimation unit 362 performs scattered ray estimation processing on the photographed image subjected to the grid stripe reduction processing, and derives a scattered ray estimation image that is an image indicating a scattered dose. In the scattered ray estimation processing in this embodiment, the scattered dose is estimated based on the relation between a photographed image, a primary radiation image and a scattered ray image, that are expressed using the primary X-ray transmittance and the scattered ray transmittance indicated by the imaging grid feature information. The obtained scattered ray estimation image is used by the scattered ray adjusting unit 363. In the scattered ray estimation processing, the scattered ray image is obtained based on a relational formula expressed by Expression 1 below, using an iterative method of the maximum-likelihood method, the least-squares method, or the like.

M(x,y)=αᵤ·P(x,y)+βᵤ·S(x,y) (1)

In Expression 1, x and y respectively indicate coordinates on the X axis and the Y axis in an image, P indicates a primary radiation image (hereinafter, primary X-ray image), S indicates a scattered ray image, M indicates a photographed image taken using an imaging grid, αᵤ indicates the primary X-ray transmittance of the imaging grid, and βᵤ indicates the scattered ray transmittance of the imaging grid.

A method for estimating a scattered ray image S while connecting a primary X-ray image P using the maximum-likelihood method based on Expression 2 below will be described as an example of the iterative method.

$${P}^{n + 1} ( x , y ) = {P}^{n} ( x , y ) \cdot \frac{M ( x , y )}{{α}_{u} \cdot {P}^{n} ( x , y ) + {β}_{u} \cdot {S}^{n} ( x , y )} \qquad ( 2 )$$

In Expression 2, Pⁿ indicates a primary X-ray image at the time of an n-th process in the iterative processing, Sⁿ indicates a scattered ray image the time of the n-th process in the iterative processing, M indicates a grid photographed image, αᵤ indicates the primary X-ray transmittance of an imaging grid, and βᵤ indicates the scattered ray transmittance of the imaging grid.

The grid-photographed image M may be used as the initial values of the primary X-ray image Pⁿ in Expression 2, or a fixed value such as 1.0 may also be used, for example. Here, a scattered ray image Sⁿ may be obtained from the primary X-ray image Pⁿ using a scattered ray model. The pixel value Sⁿ (x, y) of the x and y coordinates in the scattered ray image Sⁿ can be obtained based on Expression 3 below, for example.

$${S}^{n} ( x , y ) = {\sum }_{j = 0}^{m - 1} {\sum }_{i = 0}^{n - 1} - {P}^{n} ( i , j ) \cdot log ( \frac{{P}^{n} ( i , j )}{Q} ) \cdot exp \{ - k \cdot ( {( x - i )}^{2} + {( y - j )}^{2} ) \} \qquad ( 3 )$$

In Expression 3, i and j respectively indicate the coordinates on the X axis and the Y axis in an image, Q indicates a pixel value corresponding to the dose of radiation that has directly reached the FPD 200, and k indicates a coefficient when the spread function of scattered rays is approximated by a Gaussian distribution.

The pixel value Q in Expression 3 corresponding to the dose of radiation that has directly reached the FPD 200 can be calculated based on an imaging condition using the NDD method or the like, for example. Note that a method for obtaining the pixel value Q is not limited to this. A configuration may also be adopted in which, for example, if a direct ray region is present in a photographed image, a pixel value of this region is used as the pixel value Q, and if no direct ray region is present, the pixel value Q is obtained by multiplying a pixel value of the photographed image by the reciprocal of a representative value of the attenuation coefficient of the object. In addition, in Expression 3, the factor “−Pⁿ (i, j)·log (Pⁿ (i, j)/Q)” is obtained by approximating the intensity of the entire scattered rays, and the factor “exp{−k·(x−i)²+(y−j)²}” is obtained by approximating the spread function of the scattered rays by a Gaussian distribution.

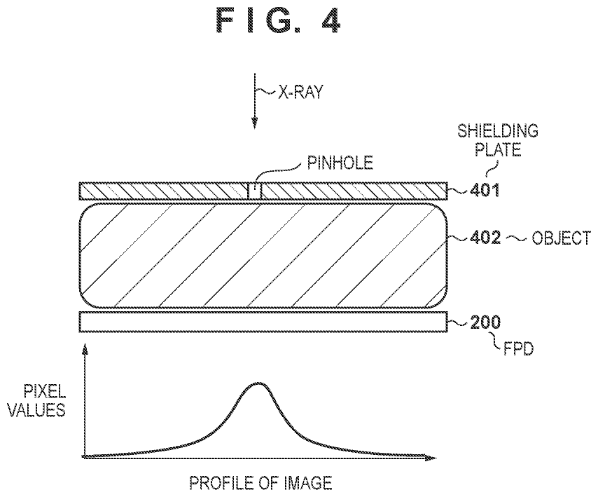

Here, an example of how to obtain the coefficient k will be described with reference to FIG. 4, When, for example, X-rays are emitted in a state where a lead plate having a very small opening therein (a shielding plate 401), an acrylic article (an object 402), and the FPD 200 are installed in the stated order from the X-ray radiation direction, the profile of a scattered image obtained from the FPD 200 is approximated, whereby the coefficient k can be obtained. The dose of scattered rays is proportional to the dose of radiation, but the scattered shape of scattered rays does not change. Therefore, by performing normalization using the pixel values of X-rays that have passed through the very small opening in the shielding plate 401 in the state where the object 402 is removed, it is possible to achieve the coefficient k that is independent on the radiation dose. Note that a configuration may also be adopted in which, since the coefficient k of the spread function changes according to the thickness of the object 402 and the tube voltage, a correspondence table is created, and the coefficient k for each condition during imaging is used. In this case, the user inputs the thickness of the object 1 from the operation unit 500, for example. The image processing apparatus 300 refers to the correspondence table using the input thickness and the tube voltage obtained from imaging information stored in the image/imaging information storage unit 305, and obtains the coefficient k.

After obtaining the terms of Expression 2, the scattered ray estimation unit 362 iterates processing until the primary X-ray image P converges using the maximum-likelihood method. A method for determining that the image has converged if the value does not change to 10 places of decimals, for example, a method for obtaining the number of times of iteration required for converge in a preliminary experiment, and determining that the image has converged when iterative processing is executed the obtained number of times of iteration, or the like can be used for this determination as to whether or not the image has converged. The scattered ray estimation unit 362 passes the scattered ray image Sⁿ when it is determined that the image has converged, as a scattered ray estimation image S′ to the scattered ray adjusting unit 363.

Next, the scattered ray adjusting unit 363 performs scattered ray adjustment processing using a scattered ray estimation image provided by the scattered ray estimation unit 362, the target grid feature information 351, and the imaging grid feature information 352, and generates a scattered-ray reduced image. The scattered ray adjustment processing aims to bring the contrast of the photographed image close to the contrast of the target grid. A method that uses Expression 4 will be described as an example of a specific method of the scattered ray adjustment processing.

M_{c}(x,y)=M(x,y)−E·S′(x,y) (4)

In Expression 4, x and y respectively indicate the coordinates on the X axis and the Y axis in an image, M_{c} (x, y) indicates the pixel values of x and y coordinates in a scattered-ray reduced image, and M (x, y) indicates the pixel values of x and y coordinates in a photographed image for which a grid was used. Moreover, E indicates a scattered ray reduction rate obtained based on the target grid feature and the imaging grid feature, and S′ (x, y) indicates the pixel values of x and y coordinates in a scattered ray estimation image estimated by the scattered ray estimation unit 362.

As indicated by Expression 4, the scattered-ray reduced image M_{c} can be obtained by subtracting, from the photographed image M, a value obtained by multiplying the scattered ray estimation image S′ by the scattered ray reduction rate E obtained based on the target grid feature and the imaging grid feature. Here, the scattered ray reduction rate E refers to a ratio for bringing the contrast of the scattered-ray reduced image M_{c} close to a contrast of imaging that uses the target grid. Methods that use Expressions 5 and 6 will be described as exemplary specific methods.

$$E = 1 - \frac{{β}_{t} \cdot {α}_{u}}{{α}_{t} \cdot {β}_{u}} \qquad ( 5 )$$

In Expression 5, E indicates a scattered ray reduction rate, αₜ and βₜ respectively indicate the primary X-ray transmittance and the scattered ray transmittance of the target grid, and αᵤ and βᵤ respectively indicate the primary X-ray transmittance and the scattered ray transmittance of the imaging grid.

Expression 5 represents an formula for calculating the scattered ray reduction rate E. This Expression 5 is a formula obtained by transforming Expression 6 below that is an equality formula of the contrast of the target grid and the contrast of the imaging grid with respect to the scattered ray reduction rate E.

log(Mₜ(x₁,y₁))−log(Mₜ(x₂,y₂))=log(M′(x₁,y₁))−log(M′(x₂,y₂)) (6)

In Expression 6, x₁ and x₂ indicate coordinates on the X axis in an image, and y₁ and y₂ indicate coordinates on the Y axis in the image. In addition, Mₜ (x, y) indicates the pixel values of x and y coordinates in a target grid image that may be obtained when the target grid is used, and M′ (x, y) indicates the pixel values of x, y coordinates of a scattered-ray reduced image that may be obtained when ideal scattered ray reduction processing is performed.

Expression 6 expresses the difference between the pixel values of two different pixels in an image obtained by performing logarithmic conversion on each of the target grid image M (left side) and the scattered-ray reduced image M′ (right side). Logarithmic conversion is performed since an image obtained by performing logarithmic conversion on an image obtained on the FPD 200 is used as an ordinary X-ray diagnosis image that is observed by the user. It can be said that, regarding the two terms of Expression 6, the difference between the two different pixels in each image indicates the contrast (difference in luminance) of the image. That is to say, the two terms being equal as is the case with Expression 6 indicates a state where the contrast in the target grid image and the contrast of the scattered ray reduction processing image are equal.

Next, Mₜ and M′ in Expression 6 will be described using Expressions 7 and 8 below. Mₜ in Expression 6 is expressed by Expression 7 below, and M′ in Expression 6 is expressed by Expression 8 below

Mₜ(x,y)=αₜ·P(x,y)+βₜ·S(x,y) (7)

In Expression 7, Mₜ (x, y) indicates the pixel values of x and y coordinates in the target grid image. In addition, P (x, y) indicates the pixel values of x and y coordinates in the primary X-ray image before X-rays reach the grid, and S (x, y) indicates the pixel values of x and y coordinates in the scattered ray image before scattered rays reach the grid. That is to say, the image P is an image of primary X-ray components before X-rays reach the grid, and the image S is an image of scattered ray components before scattered rays reach the grid. In addition, αₜ and βₜ respectively indicate the primary X-ray transmittance and the scattered ray transmittance of the target grid.

Expression 7 indicates that the target grid image Mₜ is obtained by multiplying the primary X-ray image P before X-rays reach the target grid and the scattered ray estimation image S before scattered rays reach the target grid by the primary X-ray transmittance αₜ and the scattered ray transmittance βₜ of the target grid feature, respectively, and adding the products to each other.

M′(x,y)=αᵤ·P(x,y)+(1−E)·βᵤ·S(x,y) (8)

In Expression 8, M′ (x, y) indicates the pixel values of x and y coordinates in a scattered-ray reduced image that may be obtained when ideal scattered ray reduction processing is performed. In addition, P (x, y) indicates the pixel values of x and y coordinates in a primary X-ray image before primary X-rays reach the grid, and S (x, y) indicates the pixel values of x and y coordinates in a scattered ray image before scattered rays reach the grid. Furthermore, αᵤ and βᵤ respectively indicate the primary X-ray transmittance and the scattered ray transmittance of the imaging grid, and E indicates the scattered ray reduction rate expressed by Expression 5.

Expression 8 indicates that the scattered-ray reduced image M′ is obtained by adding a term obtained by multiplying the primary X-ray image P before X-rays reach the grid by the primary X-ray transmittance αᵤ of the imaging grid to a term obtained by multiplying the scattered ray image S before scattered rays reach the grid by the scattered ray transmittance βᵤ of the imaging grid. Expression 5 is obtained by transforming Expression 8 with respect to the scattered ray reduction rate E using Expressions 6 to 8. The scattered ray adjusting unit 363 performs processing shown in Expression 4 using the scattered ray reduction rate E shown in Expression 5, thereby obtaining the scattered-ray reduced image M_{c} subjected to scattered ray adjustment processing performed to achieve a contrast equivalent to a contrast that is obtained when the target grid is used. That is to say, the scattered ray adjusting unit 363 obtains the scattered-ray reduced image M_{c} based on Expression 9 below.

$${M}_{c} ( x , y ) = M ( x , y ) - ( 1 - \frac{{β}_{t} \cdot {α}_{u}}{{α}_{t} \cdot {β}_{u}} ) \cdot {S}^{′} ( x , y ) \qquad ( 9 )$$

As described above, according to an embodiment of the present invention, it is possible to perform scattered ray estimation processing using primary X-ray transmittance and scattered ray transmittance, and generate a scattered-ray reduced image having a contrast equivalent to the contrast of a target grid. That is to say, according to the present embodiment, a scattered-ray reduced image having a contrast equivalent to the contrast of a target grid is generated based on target grid feature information and imaging grid feature information each consisting of primary X-ray transmittance and scattered ray transmittance, and a photographed image. Primary X-ray transmittance and scattered ray transmittance are basic features defined in IEC60627Ed2, and it is possible to realize scattered ray reduction processing performed on a photographed image using these basic features, making it easy to handle X-ray imaging that uses various imaging grids.

According to the present disclosure, it is possible to easily use a large variety of imaging grids along with scattered ray reduction processing.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing apparatus comprising:
a first obtaining unit configured to obtain target grid feature information indicating primary radiation transmittance and scattered ray transmittance of a target grid, and imaging grid feature information indicating primary radiation transmittance and scattered ray transmittance of an imaging grid that is used for imaging;
a second obtaining unit configured to obtain a photographed image obtained through radiography that uses the imaging grid;
an estimating unit configured to estimate a scattered dose based on a relation between the photographed image, a primary radiation image, and a scattered ray image, the relation being expressed using the primary radiation transmittance and the scattered ray transmittance indicated by the imaging grid feature information; and
an adjusting unit configured to adjust a scattered dose of the photographed image based on the scattered dose estimated by the estimating unit, the target grid feature information, and the imaging grid feature information.

2. The image processing apparatus according to claim 1,
wherein the estimating unit estimates the scattered ray image using an iterative method that uses a relational formula indicating the relation.

3. The image processing apparatus according to claim 1,
wherein, when the primary radiation image is indicated by P, the scattered ray image is indicated by S, the photographed image is indicated by M, the primary radiation transmittance of the imaging grid is indicated by αᵤ, and the scattered ray transmittance of the imaging grid is indicated by βᵤ, the relation is expressed by the following relational formula.
M(x,y)=αᵤ·P(x,y)+βᵤ·S(x,y)

4. The image processing apparatus according to claim 1,
wherein, when a primary radiation image at the time of an n-th process in iterative processing is indicated by Pⁿ, a scattered ray image at the time of the n-th process in the iterative processing is indicated by Sⁿ, the photographed image is indicated by M, and the primary X-ray transmittance and the scattered ray transmittance of the imaging grid are respectively indicated by αᵤ and βᵤ, the estimating unit estimates a scattered ray image using the following formula as a relational formula expressing the relation, using a maximum-likelihood method.
$${P}^{n + 1} ( x , y ) = {P}^{n} ( x , y ) \cdot \frac{M ( x , y )}{{α}_{u} \cdot {P}^{n} ( x , y ) + {β}_{u} \cdot {S}^{n} ( x , y )}$$

5. The image processing apparatus according to claim 4,
wherein the scattered ray image is obtained based on a scattered ray model obtained by approximating a spread of a scattered ray using a predetermined function, and the primary radiation image (Pⁿ) at the time of the iterative processing.

6. The image processing apparatus according to claim 5,
wherein the predetermined function is a Gaussian distribution.

7. The image processing apparatus according to claim 1,
wherein the adjusting unit adjusts the scattered dose so as to obtain a contrast that is close to a contrast of an image that is obtained when the target grid is used, and generates a scattered-ray reduced image.

8. The image processing apparatus according to claim 7,
wherein, when the scattered-ray reduced image is indicated by M_{c}, the photographed image is indicated by M, the primary radiation transmittance and the scattered ray transmittance of the target grid feature information are respectively indicated by αₜ and βₜ, the primary radiation transmittance and the scattered ray transmittance of the imaging grid feature information are respectively indicated by αᵤ and βᵤ, and a scattered ray estimation image that is a result of the estimating unit estimating a scattered dose is indicated by S′ (x,y), the adjusting unit generates a scattered-ray reduced image in which a scattered dose is adjusted using the following formula.
$${M}_{c} ( x , y ) = M ( x , y ) - ( 1 - \frac{{β}_{t} \cdot {α}_{u}}{{α}_{t} \cdot {β}_{u}} ) \cdot {S}^{′} ( x , y )$$

9. The image processing apparatus according to claim 1, further comprising:
an operating unit configured to accept a user operation of inputting the target grid feature information.

10. The image processing apparatus according to claim 1, further comprising:
a storage unit configured to store a correspondence between a type of imaging grid and target grid feature information,
wherein the first obtaining unit obtains target grid feature information from the storage unit based on a type of imaging grid that is used for the radiography.

11. The image processing apparatus according to claim 1, further comprising:
a storage unit configured to store a correspondence between an imaging portion and target grid feature information,
wherein the first obtaining unit obtains target grid feature information from the storage unit based on an imaging portion of the radiography.

12. The image processing apparatus according to claim 1, further comprising:
a processing unit configured to perform grid stripe reduction processing on the photographed image obtained by the second obtaining unit.

13. An image processing method comprising:
obtaining target grid feature information indicating primary radiation transmittance and scattered ray transmittance of a target grid, and imaging grid feature information indicating primary radiation transmittance and scattered ray transmittance of an imaging grid that is used for imaging;
obtaining a photographed image obtained through radiography that uses the imaging grid;
estimating a scattered dose based on a relation between the photographed image, a primary radiation image, and a scattered ray image, the relation being expressed using the primary radiation transmittance and the scattered ray transmittance indicated by the imaging grid feature information; and
adjusting a scattered dose of the photographed image based on the estimated scattered dose, the target grid feature information, and the imaging grid feature information.

14. A non-transitory computer readable storage medium storing a program for causing a computer to execute an image processing method according to claim 13.

15. An image processing apparatus comprising:
an obtaining unit configured to obtain a scattered dose of a photographed image obtained through radiography that uses an imaging grid, based on (a) target grid feature information indicating primary radiation transmittance and scattered ray transmittance of a target grid, and (b) imaging grid feature information indicating primary radiation transmittance and scattered ray transmittance of the imaging grid used for imaging.

16. An image processing method comprising:
obtaining a scattered dose of a photographed image obtained through radiography that uses an imaging grid, based on (a) target grid feature information indicating primary radiation transmittance and scattered ray transmittance of a target grid, and (b) imaging grid feature information indicating primary radiation transmittance and scattered ray transmittance of the imaging grid used for imaging.

17. A non-transitory computer readable storage medium storing a program for causing a computer to execute an image processing method according to claim 16.
